(19) United States
(12) Patent Application Publication
(10) Pub. No.: US 2026/0207510 A1
(43) Pub. Date: Jul. 23, 2026

(54) LYOPHILIZED HYDROXYUREAMETHYL ACYLFULVEN

(71) Applicant: Lantern Pharma Inc., Dallas, TX (US)

(72) Inventor: Harry Kochat, San Antonio, TX (US)

(73) Assignee: Lantern Pharma Inc., Dallas, TX (US)

(21) Appl. No.: 19/569,622

(22) Filed: Mar. 17, 2026

Related U.S. Application Data
(63) Continuation of application No. PCT/US2024/047149, filed on Sep. 18, 2024.
(60) Provisional application No. 63/538,909, filed on Sep. 18, 2023.

(51) Int. Cl. A61K 9/19; A61K 31/17; A61K 47/26; A61K 47/40

(52) U.S. Cl. CPC A61K 9/19; A61K 31/17; A61K 47/26; A61K 47/40

(57) ABSTRACT

Lyophilized preparations include illudins, including hydroxyureamethyl acylfulvene, and pharmaceutically acceptable salts thereof, in combination with cyclodextrins. These formulations are designed to enhance the solubility, stability, and therapeutic efficacy of illudins, which are known for their potent anticancer properties. Methods for producing these lyophilized preparations, including freeze-drying processes, are provided.

### CROSS-REFERENCE TO RELATED APPLICATIONS

This is application a continuation of International Patent Application No. PCT/US2024/047149, filed on Sep. 18, 2024, claims the benefit of U.S. Provisional Patent Application No. 63/538,909, filed Sep. 18, 2023, each of which is incorporated by reference herein in its entirety.

### TECHNICAL FIELD

This disclosure relates to lyophilized preparations or compositions of illudins (e.g., illudin analogs) and methods of making and using them, e.g., as cancer therapeutics.

### BACKGROUND

The use of lyophilized (freeze-dried) medicines can be beneficial in cancer treatment or management. Lyophilization preserves purity, enhances shelf life, and stabilizes various pharmaceutical products, including drugs and vaccines that are relatively unstable in solution or contain thermally unstable active ingredients. Lyophilized therapeutics are easier to reconstitute in pharmacies prior to patient administration. Careful selection of excipients for lyophilization, based on the active pharmaceutical ingredient's characteristics, allows lipophilic active molecules to be reconstituted without losing concentration due to potential precipitation. Lyophilization has also proven effective in enhancing drug stability.

Lyophilization is widely used in various industries. In pharmaceuticals, it preserves sensitive drugs, vaccines, and diagnostic reagents. In the food industry, it preserves fruits, vegetables, instant coffee, and herbs. It is also used to preserve historical documents and biological samples. Lyophilization freezes materials and removes the frozen component through sublimation, extending the shelf life of biological materials, food, and pharmaceuticals by eliminating moisture and maintaining structural integrity.

Illudin family of compounds when formulated in organic or mixture of organic and water media are unstable at room temperature storage and transportation. Illudins and illudin analogs (e.g., hydroxyureamethyl acylfulvene) pose challenges in lyophilization due to their intrinsic chemical properties. Illudins exhibit limited solubility in water, making formulation into aqueous solutions for lyophilization difficult, as they tend to precipitate or form unstable suspensions. Furthermore, illudins can degrade under certain conditions like heat, light, or pH changes. The complex chemical structure of illudins, including hydroxyureamethyl acylfulvene, with multiple hydroxyl groups and a spirocyclic ring system, can lead to interactions with other molecules or surfaces during lyophilization.

Not all drugs can be lyophilized. Some drugs may not be stable during freeze-drying or may undergo chemical or physical changes that render them ineffective or unsafe. Additionally, some drugs may have specific formulation requirements or be in a form that makes lyophilization challenging. Liposomal formulation is one of such classic examples.

Accordingly, there is a need for lyophilized formulations of illudins. It is to this need among others that this application is directed.

### SUMMARY

The applicant has surprisingly and unexpectedly discovered that illudins or their pharmaceutically acceptable salts can be lyophilized. This application is directed to lyophilized preparations comprising (a) illudins or illudin analogs (e.g., hydroxyureamethyl acylfulvene) or a pharmaceutically acceptable salt thereof; and a cyclodextrin.

One aspect includes a lyophilized drug compound having an illudin, mannitol, water, cyclodextrin, and an organic solvent. This formulation creates a stable, lyophilized product. The illudin is the active pharmaceutical ingredient, mannitol acts as a cryoprotectant and bulking agent, and cyclodextrin stabilizes by forming complexes with the illudin, improving its solubility and stability. The organic solvent aids in dissolving components before freeze-drying, ensuring even ingredient distribution. In one example, the composition includes Betadex Sulfobutyl Ether Sodium.

Another aspect includes a lyophilized drug compound where mannitol is present in an amount ranging from about 0.2% to 0.4% by weight, serving as both a bulking agent and cryoprotectant.

Another aspect includes a lyophilized drug compound with water content of about 0.1% to 3% by weight. This residual water helps balance product stability and reconstitution ease.

Another aspect includes a lyophilized drug compound with cyclodextrin in an amount of about 50% to 90% by weight, enhancing illudin solubility and stability.

Another aspect includes a lyophilized drug compound containing an organic solvent selected from alcohols, ethers, esters, ketones, and hydrocarbons, facilitating dissolution of the active drug and other ingredients before freeze-drying.

Another aspect includes a lyophilized drug compound with an organic solvent present in an amount ranging from about 50% to 90% by weight, ensuring complete dissolution of formulation components.

Another aspect includes a lyophilized drug compound using pharmaceutically acceptable grade mannitol, meeting regulatory standards for safety and efficacy.

Another aspect includes a lyophilized drug compound that may include additional pharmaceutically acceptable excipients to improve stability, solubility, and overall performance.

Another aspect includes a lyophilized composition that may include pharmaceutically acceptable excipients such as sodium phosphate, potassium phosphate, citric acid, tartaric acid, gelatin, glycine, mannitol, lactose, sucrose, maltose, glycerin, dextrose, dextran, trehalose, hetastarch, or combinations thereof, enhancing stability, protecting against degradation, and optimizing reconstitution.

Another aspect includes a method for preparing the lyophilized drug compound involving dissolving the drug compound in the organic solvent, followed by adding mannitol, water, and cyclodextrin to the drug solution. The mixture is then freeze-dried, removing water content under controlled vacuum conditions, resulting in a stable lyophilized product.

Another aspect includes a method including adding pharmaceutically acceptable excipients to the drug solution before freeze-drying to ensure optimal stability and functionality.

Another aspect includes a lyophilized drug compound having hydroxyureamethyl acylfulvene, a derivative of illudin known for its potent anticancer properties, formulated within the lyophilized product to enhance its therapeutic efficacy and stability.

### BRIEF DESCRIPTION OF THE FIGURES

FIG. 1 illustrates the lyophilization history curves for (−)-hydroxyureamethyl acylfulvene (LP-184).

FIG. 2 presents the results of long-term stability testing for LP-184.

FIG. 3 provides further results from long-term stability testing for LP-184.

FIG. 4 displays the results from accelerated stability testing for LP-184.

FIG. 5 shows additional results from long-term stability testing for LP-284.

FIG. 6 presents further results from accelerated stability testing for LP-284.

### DETAILED DESCRIPTION

Methods, compositions, and kits for providing solutions and lyophilized formulations of illudins (including illudin analogs) are disclosed herein. Illudins are often poorly soluble in water, resulting in low pharmacologically relevant concentrations. The illudin family of compounds, when formulated in organic media or a mixture of organic and aqueous media, is unstable during room temperature storage and transportation. The methods, compositions, and kits provided here offer pharmaceutically acceptable formulations, including solutions and lyophilized preparations, that address the solubility and stability issues typically encountered with such compounds. Specifically, this invention relates to lyophilized preparations comprising (a) hydroxyureamethyl acylfulvene or a pharmaceutically acceptable salt thereof, mannitol, and a cyclodextrin. Methods for preparing and using these lyophilized preparations are also described.

Illudins and their analogs pose challenges in lyophilization due to their intrinsic chemical properties. Illudins exhibit limited solubility in water, making it difficult to formulate them into stable aqueous solutions for lyophilization, as they tend to precipitate or form unstable suspensions. Furthermore, illudins are sensitive to degradation under conditions such as heat, light, and pH variations. The complex chemical structure of illudins, including hydroxyureamethyl acylfulvene, which has multiple hydroxyl groups and a spirocyclic ring system, can lead to interactions with other molecules or surfaces during the lyophilization process.

The term “illudin” can refer to several different compounds, commonly describing a group of naturally occurring toxins produced by certain fungi in the Omphalotus genus and related genera. These toxins are known for their extreme toxicity and have been studied for their potential applications in cancer therapy. Despite their toxicity, illudins have been researched for their potential as anticancer agents, with studies investigating their ability to selectively target and kill cancer cells. Some studies suggest that illudins may have potential as chemotherapeutic agents, particularly in the treatment of specific types of cancer.

Illudins are characterized by a unique chemical structure consisting of a fused six-membered ring with a five-membered ring. This structural feature contributes to their biological activity. Analog compounds are chemical variants or derivatives of the parent illudin compound that have been modified to potentially enhance their properties or reduce toxicity. In this context, “illudins” and their analogs refer to a group of chemical compounds with possible medicinal or therapeutic properties.

### Illudin or Acylfulvene

One embodiment of this application includes the use of an illudin or illudin analog, such as acylfulvene. Acylfulvene analogs are a class of cytotoxic, semi-synthetic derivatives of illudin, which can be extracted from the jack-o'-lantern mushroom (Omphalotus olearius). Acylfulvene derivatives, derived from the sesquiterpene illudin S through a reverse Prins reaction with acid, are far less reactive to thiols than illudin S.

In one example, the acylfulvene analog is (−)-hydroxyureamethyl acylfulvene (termed LP-184 by Lantern Pharma Inc.), which shifts light negatively, is shown below:

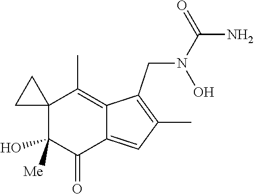 [SMILES: CC1=C(CN(O)C(N)=O)C2=C(C)C3(CC3)[C@@](C)(O)C(=O)C2=C1]

In another example, the acylfulvene analog is (+)-hydroxyureamethyl acylfulvene (termed LP-284 by Lantern Pharma Inc.), which shifts light positively, is shown below:

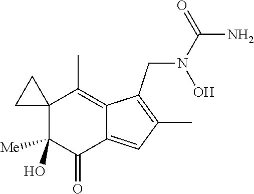 [SMILES: CC1=C(CN(O)C(N)=O)C2=C(C)C3(CC3)[C@](C)(O)C(=O)C2=C1]

Both (+)-hydroxyureamethyl acylfulvene and (−)-hydroxyureamethyl acylfulvene are enantiomers that are now publicly known.

Another example includes hydroxymethyl acylfulvene, also known as Irofulven.

Another embodiment includes lyophilized preparations comprising (a) hydroxyureamethyl acylfulvene or a pharmaceutically acceptable salt thereof, and Betadex Sulfobutyl Ether Sodium. Methods of preparing and using these lyophilized preparations are also described.

In other embodiments, various polymers can be used as alternatives to cyclodextrins in lyophilized drugs. Examples include polyvinylpyrrolidone (PVP), a water-soluble polymer that can improve the solubility of hydrophobic compounds; hydroxypropyl methylcellulose (HPMC), a cellulose-based polymer that can serve as a cryoprotectant and stabilizer during lyophilization; or dextran, a polysaccharide used to enhance the stability of lyophilized formulations, particularly for proteins and peptides.

One embodiment includes the use of Betadex Sulfobutyl Ether Sodium, also known as sulfobutyl ether β-cyclodextrin sodium (SBE-β-CD), a chemically modified derivative of beta-cyclodextrin. SBE-β-CD is widely used in pharmaceutical formulations as a complexing agent to improve the solubility, stability, and bioavailability of certain drug compounds. Cyclodextrins, which are cyclic oligosaccharides with a hydrophobic interior cavity and a hydrophilic outer surface, form inclusion complexes with various drug molecules.

Another embodiment includes hydroxyureamethyl acylfulvene formulated as a lyophilized powder for injection. The product is reconstituted as close to the time of patient administration as possible. For the 15 mg product, reconstitution is achieved by aseptically adding 5 mL of Sterile Water for Injection, USP (SWFI). For the 100 mg product, 20 mL of SWFI is used. The vial is shaken thoroughly, and complete dissolution should occur within 5 minutes. After dissolution in SWFI, the required volume for the appropriate dose is aseptically withdrawn and immediately transferred to a 500 mL infusion bag containing 0.9% Sodium Chloride Injection USP (normal saline) or 2.5% Dextrose/0.45% Sodium Chloride Injection USP. Transfer to the infusion bag must be completed within 30 minutes of reconstitution to ensure product stability and efficacy.

This invention also applies to the pharmaceutically acceptable salts of hydroxyureamethyl acylfulvene. “Pharmaceutically acceptable salts” refer to derivatives of the disclosed compounds, wherein the parent compound is modified by forming acid or base salts. These salts may include, but are not limited to, mineral or organic acid salts of basic residues (such as amines) or alkali or organic salts of acidic residues (such as carboxylic acids). Pharmaceutically acceptable salts include non-toxic salts or quaternary ammonium salts formed from non-toxic inorganic or organic acids. Preferred examples include salts derived from hydrochloric, hydrobromic, sulfuric, phosphoric, citric, and other pharmaceutically acceptable acids.

Specific embodiments encompass lyophilized preparations of hydroxyureamethyl acylfulvene or its pharmaceutically acceptable salts in combination with a cyclodextrin. Cyclodextrins are cyclic oligosaccharides containing five or more α-D-glucopyranoside units, which can enhance the solubility and stability of many drugs, including hydroxyureamethyl acylfulvene. The cyclodextrins of this invention include naturally occurring and derivatized forms, such as hydroxypropyl-β-cyclodextrin or sulfobutyl ether β-cyclodextrin, which are preferred for their solubilizing properties.

Cyclodextrin derivatives such as methyl-β-cyclodextrin, dimethyl-β-cyclodextrin, hydroxypropyl-β-cyclodextrin, and sulfobutyl ether β-cyclodextrin are particularly effective in stabilizing hydroxyureamethyl acylfulvene during and after lyophilization. These cyclodextrins can be present in amounts up to 99% w/w of the lyophilized preparation, with preferred concentrations ranging from 20% to 90% w/w. The most preferred embodiments contain 40% to 70% w/w cyclodextrin.

In addition to cyclodextrins, the lyophilized preparation may also contain bulking agents to enhance the structure of the resulting cake. Bulking agents such as mannitol are commonly used in pharmaceutical lyophilization processes to strengthen the lyophilized product, making it more stable and easier to handle. Mannitol may be present in concentrations ranging from 20% to 60% w/w of the lyophilized preparation.

In some embodiments, the pre-lyophilization solution is prepared using a pharmaceutically acceptable organic solvent, such as ethanol, tert-butanol, acetone, ethyl acetate or heptane. These solvents assist in solubilizing hydroxyureamethyl acylfulvene, making the lyophilization process more efficient. After lyophilization, residual amounts of the solvent are reduced to less than 3% w/w, ensuring the safety and efficacy of the final product.

The lyophilized formulations can be reconstituted quickly, forming clear, colorless solutions free of particulate matter in under 180 seconds. In some preferred embodiments, reconstitution occurs in less than 60 seconds, providing a significant improvement over existing formulations.

The lyophilized cake of hydroxyureamethyl acylfulvene can contain varying concentrations of the active ingredient, depending on the intended therapeutic use and formulation requirements. Typically, the concentration of hydroxyureamethyl acylfulvene in the lyophilized cake ranges from about 2.5 mg/mL to 30 mg/mL or 2.5 mg/ml to 25 mg/ml. In one example, the cake has 15 mg/ml. This range allows for flexibility in dosing while maintaining the stability and efficacy of the drug. The concentration can be controlled to ensure that the lyophilized cake is stable throughout its shelf life and can be easily reconstituted for administration. In specific formulations, the concentration can be adjusted based on the desired therapeutic effect, the solubility of the active ingredient, and compatibility with excipients such as mannitol and cyclodextrin. These excipients play a role in maintaining the structural integrity of the lyophilized cake while enhancing the solubility and stability of hydroxyureamethyl acylfulvene.

These lyophilized preparations may also be administered in combination with other antineoplastic agents. Combination therapies are widely used in the treatment of neoplastic diseases, and hydroxyureamethyl acylfulvene can be co-administered with agents such as glucocorticoids, interferons, or other anticancer drugs to enhance therapeutic efficacy.

This invention thus provides a significant advancement in the formulation of hydroxyureamethyl acylfulvene, improving its solubility, stability, and ease of use for clinical applications.

One embodiment includes a method for manufacturing this solid composition of lyophilized hydroxyureamethyl acylfulvene, which involves dissolving hydroxyureamethyl acylfulvene in an organic lyophilization solvent and subjecting the solution to a first lyophilization. The resulting cake, containing up to 8% w/w organic lyophilization solvent, is then reconstituted with an aqueous solution and subjected to a second lyophilization. The method can include the following steps:
a) Dissolving hydroxyureamethyl acylfulvene in an organic lyophilization solvent,
b) Lyophilizing,
c) Dissolving the lyophilized hydroxyureamethyl acylfulvene from step (b) in an aqueous solution, and
d) Lyophilizing the hydroxyureamethyl acylfulvene aqueous solution from step (c).

Lyophilization offers several advantages for drug preservation and storage. By removing moisture, it minimizes the potential for microbial growth and chemical degradation, extending the shelf life of the drug. Additionally, the lyophilized product can be easily reconstituted with a suitable solvent, such as sterile water, before administration to the patient.

An optimal lyophilized cake should have a uniform appearance, adhere to the vial walls without detaching, and be free of cavities at the bottom. The color of the cake should be consistent, with slight variations possible due to the formation of crystals during the freezing process, whether dendritic or small and amorphous. In one example, a combination of mannitol and cyclodextrins provided such a cake.

Lyophilized formulations of hydroxymethyl acylfulvene typically include various excipients, such as buffers, stabilizers, bulking agents, and tonicity modifiers. A well-chosen excipient formula ensures a lyophilized product with a long shelf life and stable biological activity. Excipients play a critical role in pharmaceutical product development to achieve the desired product profile, including stability and efficacy.

One advantage of the specific embodiments is that they provide a simple and stable lyophilized formulation while maintaining acceptable chemical, physical, and microbiological stability.

In formulations with low concentrations of illudin, bulking agents help create a larger, firmer cake structure. Crystalline bulking agents are commonly used to produce well-formed cakes with good mechanical properties. Bulking agents such as mannitol and glycine are utilized in lyophilized formulations to provide structure to the lyophilized cake, preventing collapse.

Other ingredients include cryoprotectants or lyoprotectants, which are used to protect biological materials such as cells, tissues, proteins, and vaccines from freezing (cryoprotectants) or drying (lyoprotectants). These substances are essential for preserving the viability and functionality of these materials during long-term storage or transportation. Excipients such as sugars (e.g., sucrose, trehalose), sugar alcohols (e.g., mannitol, sorbitol), and surfactants (e.g., polysorbate 20, 80) are commonly used to improve stability.

### Tonicity Modifiers

The tonicity adjuster is typically selected based on the stability requirements of the bulk solution or the route of administration. Excipients like mannitol, sucrose, glycine, glycerol, and sodium chloride are commonly used as tonicity adjusters. These excipients can be included in the diluent rather than the formulation itself and play a significant role in enhancing the rapid solubility and absorption of lyophilized products.

The following examples are provided for illustrative purposes only and should not be construed as limiting the scope of the invention.

### EXAMPLES

### Example 1-Lyophilized (−)-Hydroxyureamethyl Acylfulvene (LP-184)

The lyophilized composition consists of several key components. LP-184 is the primary active ingredient, present at a concentration of 2.5 mg/mL. Mannitol is included at 83.3 mg/mL, functioning as a bulking agent to help maintain product integrity during freeze-drying, a critical part of the manufacturing process. Betadex Sulfobutyl Ether Sodium is present at a concentration of 166.7 mg/mL, acting as a complexing agent to improve solubility and stability. Water is used to bring the formulation to 99% and is later removed during the freeze-drying process to ensure a stable, dry product.

The manufacturing process for this composition involves several steps. First, Betadex Sulfobutyl Ether Sodium (BSES) is added to a tank and heated to a temperature between 6° and 65° C. Afterward, the solution is cooled to approximately 25° C. to prepare for the addition of other components. LP-184 is added, followed by the addition of Mannitol. Water is then introduced to achieve the final composition of 99%.

Once the solution is prepared, it undergoes a freeze-drying process. The temperature is lowered below 15° C. under a vacuum to remove the water content. The material is frozen for up to 240 minutes to stabilize it before primary drying occurs. During the primary drying phase, pressure is maintained between 70 and 200 mTorr, with drying cycles lasting over 1500 minutes. If necessary, a secondary drying step is performed, during which the product is dried at a higher temperature (typically 50 mTorr) for more than 360 minutes. This freeze-drying process is crucial for ensuring the stability of the lyophilized product, which can be reconstituted when needed for use.

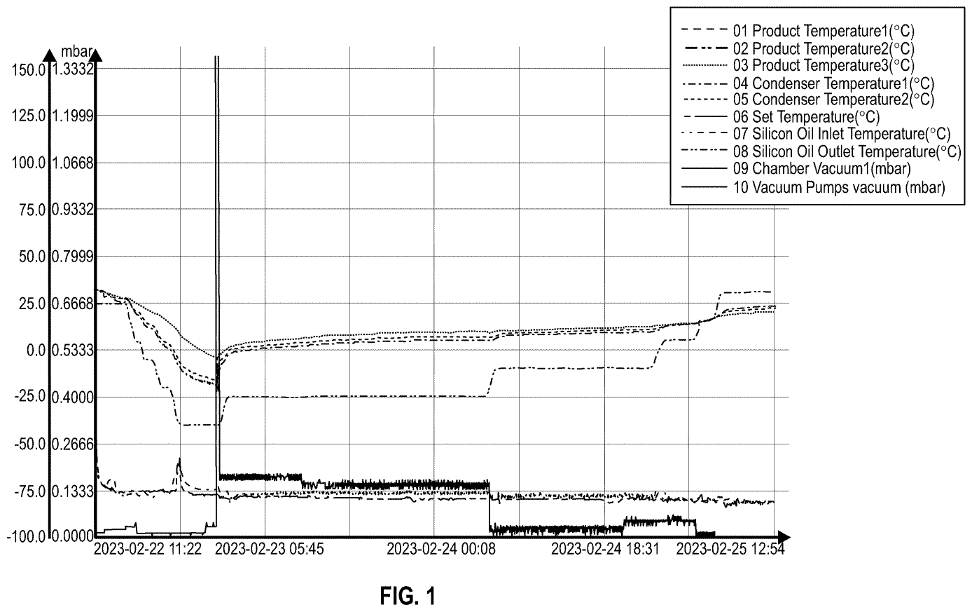

FIG. 1 shows the lyophilization history curves for (−)-hydroxyureamethyl acylfulvene. The lyophilization history curves or freeze-drying process curves are graphical representations used to monitor and control the critical stages of the freeze-drying process in pharmaceutical and biotechnological production. These curves show the relationship between time, temperature, pressure, and other parameters during the three main phases of lyophilization: freezing, primary drying, and secondary drying. They help visualize how the product, shelf, and chamber temperature evolve over time, ensuring optimal conditions and a stable final product.

### Example 2-Lyophilized (+)-Hydroxyureamethyl Acylfulvene (LP-284)

This lyophilized composition is designed to ensure stability and efficacy. LP-284, one of the active components, is present at a concentration of 2.5 mg/mL. Mannitol, included at 83.3 mg/mL, serves as a bulking agent to protect the formulation during freeze-drying, functioning as both a stabilizer and cryoprotectant. Sulfo Butyl Ether Beta Cyclodextrin, present at 166.7 mg/mL, acts as a complexing agent, enhancing the solubility and stability of LP-284. Water is added as a solvent to bring the composition up to 99%, which is later removed during freeze-drying to ensure the product's stability.

The manufacturing process for this composition involves several critical steps. The process begins by dissolving Sulfo Butyl Ether Beta Cyclodextrin in a tank at a temperature between 6° and 65° C. Once dissolved, the solution is cooled to around 25° C. in preparation for the addition of other components. LP-284 is then added, followed by the addition of Mannitol, which bulks the formulation and protects it during freeze-drying. Water is then added to reach the desired final composition.

Once the solution is prepared, it undergoes freeze-drying to remove water and stabilize the product. The solution is cooled to below 15° C., and vacuum conditions are applied to facilitate water sublimation. The material is initially frozen for up to 240 minutes, followed by primary drying under pressures ranging from 70 to 200 mTorr, which can last for over 1500 minutes. A secondary drying step may be employed to further reduce moisture content. This involves drying at a higher temperature (around 50 mTorr) for more than 360 minutes, depending on the product's stability requirements.

### Example 3: Stability of Lyophilized LP-184

As demonstrated by the 12-month stability testing conducted at both 25° C./60% RH (relative humidity) and 2-8° C., the lyophilized product of Example 1 remained stable over the course of one year. The concentration of LP-184 in the product was 15.0 mg/mL, and the formulation showed no significant degradation or loss of potency during this period. To ensure the product's reliability, the clinical batch must undergo both accelerated and long-term stability testing as part of regulatory and quality assurance requirements.

For these studies, samples are stored upright in qualified stability storage rooms and cabinets. Temperature and humidity are continuously monitored to ensure adherence to the specified conditions. Throughout the testing, the product demonstrated stability when stored at 25° C. (77° F.), with no observable changes in appearance, potency, or integrity.

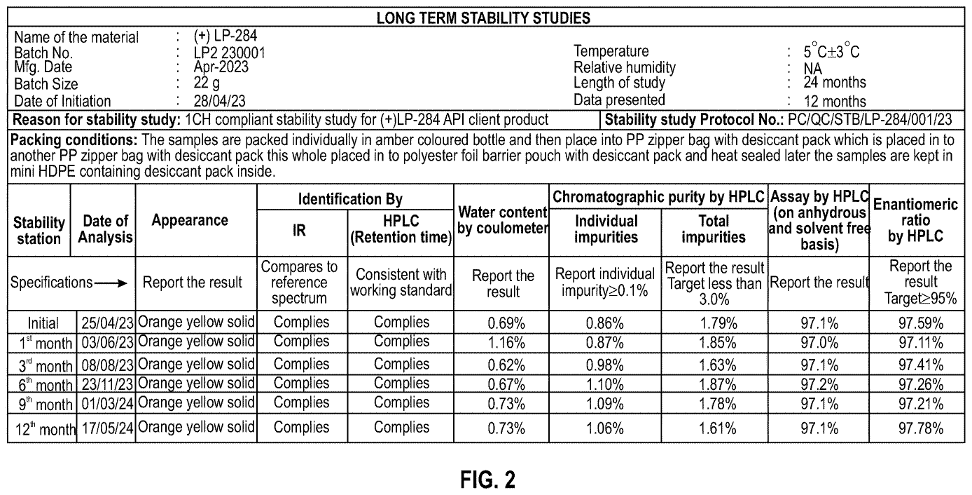

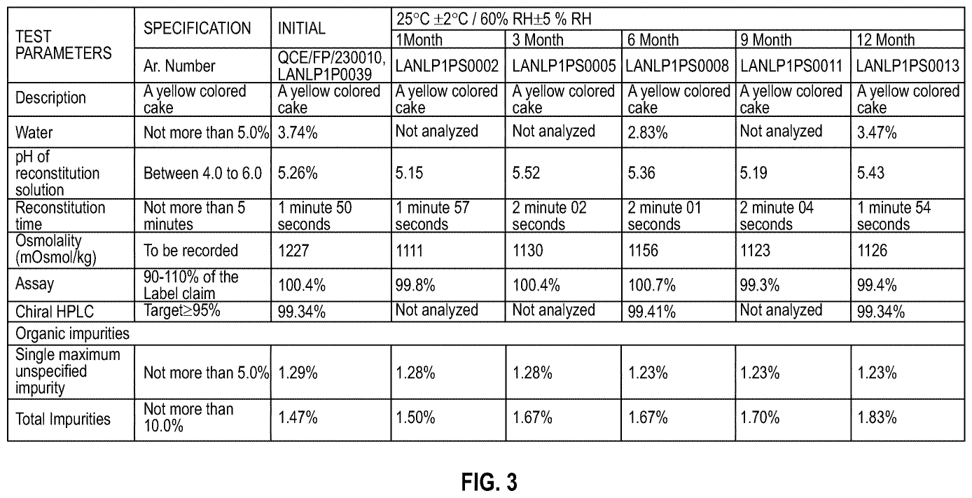

FIGS. 2 and 3 illustrate the results of the long-term stability study, showing the product's consistency under normal storage conditions. Additionally, FIG. 3 presents the results of the accelerated stability study, where the product was exposed to more extreme conditions to simulate long-term effects over a shorter period. These studies confirmed the robustness of the formulation, validating its suitability for clinical use and ensuring it meets regulatory stability requirements.

### Example 4: Stability of Lyophilized LP-284

As demonstrated by the 12-month stability testing conducted at both 25° C./60% RH and 2-8° C., the lyophilized product of Example 2 (LP-284) remained stable over a one-year period. This testing confirmed the product's ability to retain its physical and chemical properties under both standard and refrigerated storage conditions, highlighting its robustness for long-term use.

For regulatory compliance, the clinical batch of the product is required to undergo both accelerated and long-term stability testing to assess its behavior under various environmental conditions. These tests are essential to understanding how the product will perform over its shelf life and ensure it meets all safety and efficacy standards. During the testing period, samples are stored upright in qualified stability storage rooms and cabinets that are designed to maintain consistent conditions. Temperature and humidity are continuously monitored and recorded using validated systems to ensure precise control of the storage environment.

The stability testing confirmed that the product maintains its integrity and stability when stored at 25° C. (77° F.), with no significant degradation in potency, appearance, or other critical quality attributes. Ensuring the product's stability throughout its intended shelf life is vital to providing consistent therapeutic benefits.

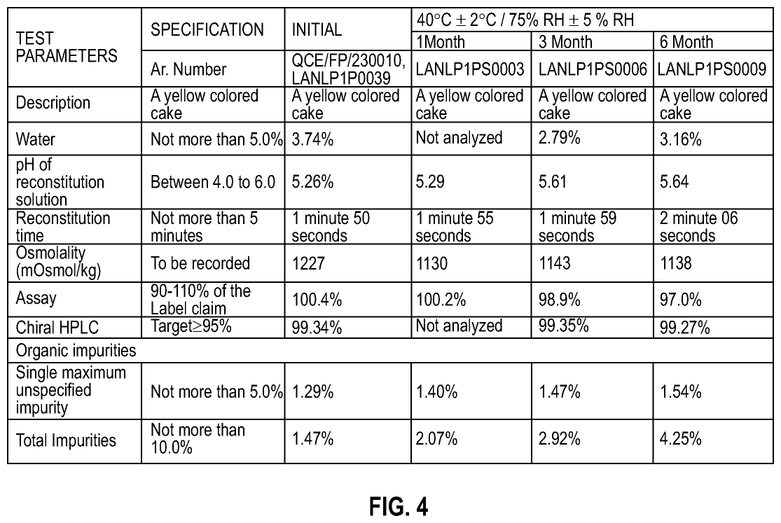

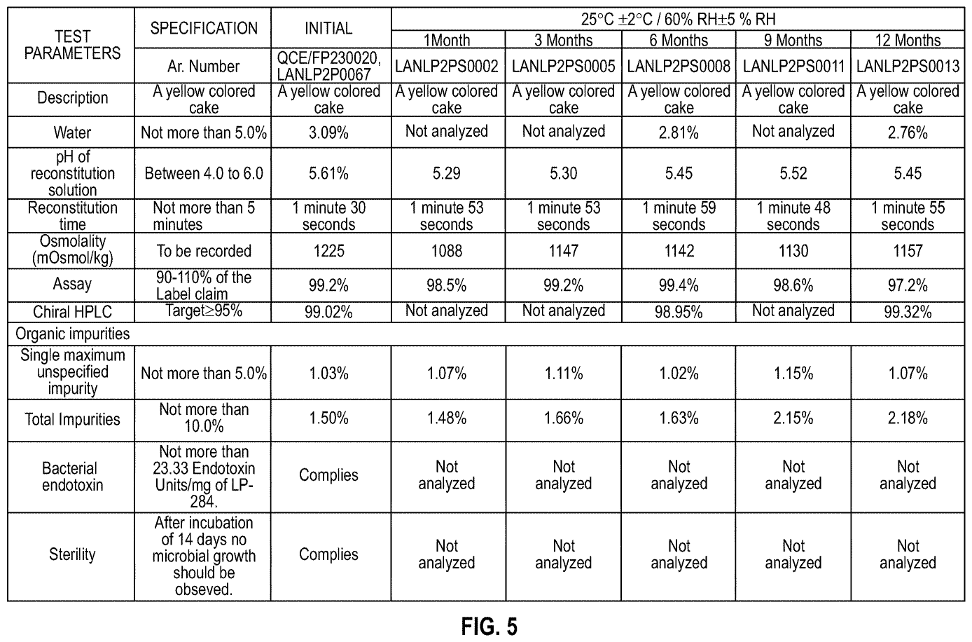

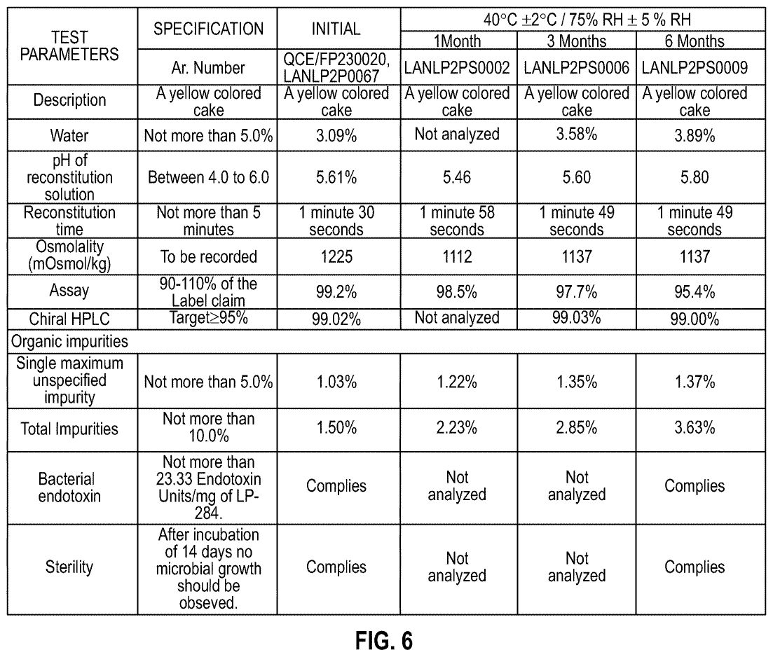

FIG. 4 outlines the results of the long-term stability study, verifying that the product's key characteristics are preserved over time. Additionally, FIG. 5 displays the results of the accelerated stability study, where the product was exposed to stressful conditions to simulate long-term storage effects within a shorter time frame. FIG. 6 presents further results from accelerated stability testing for LP-284. The findings from the accelerated study further support the product's stability, demonstrating its resilience to environmental challenges during storage and transportation. This provides confidence in the product's long-term reliability. These comprehensive stability tests are essential in ensuring the product's quality, safety, and efficacy, validating its suitability for both clinical and commercial use.

As those skilled in the art will appreciate, numerous modifications and variations of the present invention are possible in view of the above teachings. It is therefore understood that within the scope of the appended claims, the invention can be practiced otherwise than as specifically described herein, and the scope of the invention is intended to encompass all such variations.

What is claimed:

1. A lyophilized pharmaceutical composition comprising:
(a) an illudin or illudin analog, or a pharmaceutically acceptable salt thereof,
(b) a cyclodextrin; and
(c) mannitol.

2. The composition of claim 1, wherein the illudin analog is an acylfulvene.

3. The composition of claim 2, wherein the acylfulvene is hydroxyureamethyl acylfulvene, or a pharmaceutically acceptable salt thereof.

4. The composition of claim 3, wherein the hydroxyureamethyl acylfulvene is (−)-hydroxyureamethyl acylfulvene.

5. The composition of claim 3, wherein the hydroxyureamethyl acylfulvene is (+)-hydroxyureamethyl acylfulvene.

6. The composition of claim 1, wherein the cyclodextrin is selected from the group consisting of hydroxypropyl-β-cyclodextrin, sulfobutyl ether β-cyclodextrin, methyl-β-cyclodextrin, and dimethyl-β-cyclodextrin.

7. The composition of claim 6, wherein the cyclodextrin is sulfobutyl ether β-cyclodextrin sodium.

8. The composition of claim 1, wherein the cyclodextrin is present in an amount of about 20% to about 90% by weight of the lyophilized composition.

9. The composition of claim 8, wherein the cyclodextrin is present in an amount of about 40% to about 70% by weight of the lyophilized composition.

10. The composition of claim 1, wherein the mannitol is present in an amount of about 20% to about 60% by weight of the lyophilized composition.

11. The composition of claim 1, wherein the lyophilized composition comprises residual water in an amount of about 0.1% to about 3% by weight.

12. The composition of claim 1, wherein the lyophilized composition comprises residual organic solvent in an amount of less than about 3% by weight.

13. The composition of claim 12, wherein the organic solvent is selected from the group consisting of ethanol, tert-butanol, acetone, ethyl acetate, and heptane.

14. The composition of claim 3, wherein the composition is stable for at least about 12 months when stored at about 25° C./60% relative humidity or at about 2° C. to about 8° C.

15. The composition of claim 3, wherein the composition is configured to reconstitute in less than about 180 seconds to provide a clear, colorless solution substantially free of particulate matter and containing about 2.5 mg/mL to about 30 mg/mL of hydroxyureamethyl acylfulvene.

16. A method of preparing a lyophilized pharmaceutical composition comprising an illudin or illudin analog, or a pharmaceutically acceptable salt thereof, a cyclodextrin, and mannitol, the method comprising:
(a) combining the illudin or illudin analog, or pharmaceutically acceptable salt thereof, with the cyclodextrin, mannitol, and a solvent system comprising water or a mixture of water and a pharmaceutically acceptable organic solvent to form a pre-lyophilization composition; and
(b) subjecting the pre-lyophilization composition to lyophilization.

17. The method of claim 16, wherein a cyclodextrin-containing solution is heated to about 60° C. to about 65° C. and subsequently cooled to about 25° C. before addition of the illudin or illudin analog.

18. The method of claim 16, wherein the pre-lyophilization composition is frozen for up to about 240 minutes before primary drying.

19. The method of claim 16, wherein primary drying is performed at about 70 mTorr to about 200 m Torr for more than about 1500 minutes.

20. The method of claim 19, further comprising secondary drying at about 50 mTorr for more than about 360 minutes.

21. The method of claim 16, further comprising reconstituting a first lyophilized product in an aqueous solution and subjecting the reconstituted product to a second lyophilization.

22. A pharmaceutical product comprising a vial containing a lyophilized pharmaceutical composition, the composition comprising hydroxyureamethyl acylfulvene, or a pharmaceutically acceptable salt thereof, a cyclodextrin, and mannitol, wherein the composition is configured for reconstitution with sterile water for injection prior to administration to a subject.

23. The pharmaceutical product of claim 22, wherein, after reconstitution, the composition is transferred to 0.9% sodium chloride injection or 2.5% dextrose/0.45% sodium chloride injection within about 30 minutes of reconstitution.

24. A method of treating a disease in a subject, comprising:
reconstituting a lyophilized pharmaceutical composition comprising hydroxyureamethyl acylfulvene, or a pharmaceutically acceptable salt thereof, a cyclodextrin, and mannitol, in a pharmaceutically acceptable aqueous solution; and
administering a therapeutically effective amount of the reconstituted composition to the subject.

25. The method of claim 24, wherein the reconstituted composition is administered by intravenous infusion over about 30 to about 60 minutes.
